(19) United States
(12) Patent Application Publication
(10) Pub. No.: US 2016/0338715 A1
(43) Pub. Date: Nov. 24, 2016

(54) Anatomical Alignment Systems and Methods

(71) Applicant: ConforMIS, Inc., Bedford, MA (US)

(72) Inventors: Raymond A. Bojarski, Attleboro, MA (US); Paul Dietz, Charlestown, MA (US)

(21) Appl. No.: 15/225,260

(22) Filed: Aug. 1, 2016

Related U.S. Application Data
(63) Continuation of application No. 13/726,402, filed on Dec. 24, 2012, now abandoned.
(60) Provisional application No. 61/580,179, filed on Dec. 23, 2011.

(51) Int. Cl. A61B 17/17

(52) U.S. Cl. CPC A61B 17/1764

(57) ABSTRACT

Systems, methods and devices are disclosed for aligning surgical jigs, tools and implants in a patient.

### RELATED APPLICATIONS

This application is a continuation of U.S. Ser. No. 13/726,402, entitled “Anatomical Alignment Systems and Methods,” filed Dec. 24, 2012, which in turn claims the benefit of U.S. Ser. No. 61/580,179 entitled “Anatomical Alignment Systems and Methods,” filed Dec. 23, 2011. Each of these applications is hereby incorporated herein by reference in its entirety.

### TECHNICAL FIELD

The embodiments described herein relate to systems and methods for accurately aligning surgical jigs, tools and implants in a patient utilizing less-invasive and/or non-invasive alignment tools.

### BACKGROUND

Historically, diseased, injured or defective joints, including joints exhibiting osteoarthritis, have been repaired using standard off-the-shelf implants and other surgical devices. Such surgical implant systems generally employed a one-size-fits-all or a “few-sizes-fit-all” approach (including modularly assembled systems), and utilized gross anatomical measurements such as the maximum bone dimensions at the implant site, as well as the patient weight and age, to determine a “suitable” implant. The surgical procedure then concentrated on altering the underlying bony anatomical support structures (e.g., by cutting, drilling and/or otherwise modifying the bone structures) to accommodate the existing contact surfaces of the pre-manufactured implant. If the underlying anatomical measurements were inaccurate and/or incorrect, the underlying bony support structures would often be modified to accommodate implantation of the implant.

More recently, the joint replacement field has come to embrace the concept of “patient-specific” and “patient-engineered” implant systems. With such systems, the surgical implants and associated surgical tools and procedures are designed or otherwise modified to account for and accommodate the individual anatomy of the patient undergoing the surgical procedure. Such systems typically utilize non-invasive imaging data, taken of the individual pre-operatively, to guide the design and/or selection of the implant, surgical tools, and the planning of the surgical procedure itself. Various objectives of these newer systems include: (1) reducing the amount of bony anatomy removed to accommodate the implant, (2) designing/selecting an implant that replicates and/or improves the function of the natural joint, (3) increasing the durability and functional lifetime of the implant, (4) simplifying the surgical procedure for the surgeon, (5) reducing patient recovery time and/or discomfort, and (6) improving patient outcomes.

Regardless of implant selection and/or design, the preparation of the surgical site (e.g., bone and/or soft tissue structures), as well as the ultimate placement of the implant can significantly affect patient outcomes and satisfaction. A misaligned implant and/or improperly prepared anatomical site can contribute to premature wear and/or implant failure. Such implants may also be at greater risk of dislocation or separation from the underlying anatomical support structure. Moreover, the improperly aligned implant may adversely affect the kinematics of the treated joint and/or limb, which may cause unintended wear and/or damage to other anatomical structures (e.g., a malfunctioning knee may contribute to degradation of the involved hip and/or opposing knee).

Accordingly, there is a need in the art for advanced methods, techniques, devices and systems to ensure proper preparation of anatomical structures and proper alignment of implant components in the placement of orthopedic implant components.

### SUMMARY

Various embodiments described herein include systems and methods to facilitate the placement, positioning, orientation and/or alignment of surgical jigs, tools and/or implant components for performing an orthopedic implantation procedure on a patient. Some embodiments can include a docking sleeve with a proximal portion configured for connecting to one or more surgical jigs, tools, and/or implant components. A distal portion of the docking sleeve may be configured to be connected to a proximal portion of a swivel arm. A distal portion of the swivel arm can be configured for connecting to a mating arm. The swivel arm can permit controlled movement of the mating arm relative to the docking sleeve. The mating arm can include a portion configured to engage an anatomical structure, or surface adjacent thereto, that is spaced or distanced from an implantation site of interest. This engagement and any relative movement between the mating arm, swivel arm, and/or docking sleeve can be used to determine alignment of the one or more surgical jigs, tools, and/or implant components relative to an axis of the patient.

It is to be understood that the features of the various embodiments described herein are not mutually exclusive and may exist in various combinations and permutations.

### BRIEF DESCRIPTION OF THE DRAWINGS

The foregoing and other objects, aspects, features, and advantages of embodiments will become more apparent and may be better understood by referring to the following description, taken in conjunction with the accompanying drawings, in which:

FIG. 1 shows a perspective view of one embodiment of an alignment tool constructed in accordance with various teaching of the present disclosure;

FIGS. 1A through 1C show various component sections of the alignment tool of FIG. 1;

FIG. 2 shows the alignment tool of FIG. 1 attached to an alignment rod of a surgical tool engaged to a corresponding bone surface portion;

FIG. 3 shows the alignment tool of FIG. 1 engaged with an alignment rod, with various portions of the alignment tool shown in phantom;

FIG. 4 is a top plan view of a distal portion of the alignment tool of FIG. 1 with various portions of the tool shown in phantom;

FIG. 5 is a perspective view of the distal portion of alignment tool of FIG. 4;

FIG. 6 is a side perspective view of the alignment tool of FIG. 1, with various portions of the tool shown in phantom;

FIG. 7 shows a perspective view of a surgical alignment tool with an attached alignment rod;

FIG. 8 shows the surgical alignment tool of FIG. 7 with an extension rod connected to the attached alignment rod;

FIG. 9 depicts a perspective view of an alternate embodiment of an alignment tool connected to the extension rod of FIG. 8;

FIG. 10 depicts a perspective view of an alignment tool attached to an alignment rod;

FIG. 11 depicts another perspective view of the alignment tool of FIG. 10;

FIG. 12 depicts another perspective view of the alignment tool of FIG. 10;

FIG. 13A depicts a perspective view of the distal head of the alignment tool of FIG. 10;

FIG. 13B depicts a cross-sectional view of the distal head of FIG. 13A, taken along views 13B-13B;

FIGS. 13C and 13D depict perspective views of the distal head of FIG. 13A;

FIGS. 14A through 14C depict various views of an intermediate or swivel section of the alignment tool of FIG. 10;

FIGS. 15A and 15B depict views of a proximal or engagement section of the alignment tool of FIG. 10;

FIG. 16 depicts a bottom perspective view of the alignment tool of FIG. 10;

FIGS. 16A and 16B depict cross-sectional views of the alignment tool of FIG. 16, taken along views 16A-16A and 16B-16B, respectively; and

FIG. 17 depicts the alignment tool of FIG. 10 attached to an alignment rod of a surgical tool incorporating patient-specific and/or patient-adapted features.

Additional figure descriptions are included in the text below.

### DETAILED DESCRIPTION

In preparing an anatomical site for implantation of a joint resurfacing and/or replacement implant, it is often desirable and necessary to remove various portions of the patient's existing anatomy. Such removal can be for many reasons, including a desire to remove diseased, injured and/or otherwise unwanted tissues, to resect tissues to create a secure anchoring location for the implant components, and/or to remove tissues to accommodate the orientation, sizing and/or spacing of the various components within the joint.

Orthopedic implant components typically have an optimal orientation and/or position on a given anatomical support structure, which can be dictated by a wide variety of design and performance criteria, as well as the condition and suitability of the patient's anatomy. In general, the eventual positioning and orientation of the implant components is a result of the alignment of the interior-facing (i.e., bone-facing) surfaces and support structures (e.g., pegs or anchors) with the prepared surface(s) of the patient's anatomy.

In various implant systems, a desired alignment for components of a joint implant system (e.g., jigs, tools, implant components) can be relative, in one or more planes, to a mechanical, anatomical and/or biomechanical axis of one or more anatomical structures and/or opposing implant component surfaces (e.g., a bone, a joint structure, a combination of joints, an extremity, etc.). For example, in some systems, the desired alignment for components of a joint implant system can be relative to a mechanical, anatomical, and/or biomechanical axis of a bone (e.g., tibia, femur) and/or of a limb (e.g., lower extremity, upper extremity) to be treated. In such cases, a resected bone surface can be prepared with a known position and orientation which, when the resected bone surface is engaged and aligned with a bone-facing surface of the implant system component, will result in a desired position and orientation of the implant component on the patient's anatomy.

For example, a surgeon may desire to implant knee prosthesis components that are aligned relative to the mechanical axis of the patient's lower extremity. The mechanical axis of the patient's lower extremity can be established by, for example, drawing a line on an appropriate x-ray from the patient's hip to ankle when the patient is in a stable, erect attitude. In practice, this mechanical axis is generally a line or axis drawn through the longitudinal center of the patient's tibia that intersects the center of the femoral head. This axis can be unique for each particular patient, and can be referenced from the anatomic axis of the patient's femur, which is the axis through an intramedullary channel in the femur bone. In many instances, this angular difference from the vertical of the mechanical to anatomic axis can be five degrees to six degrees. Occasionally, in patients who have had total hip arthroplasty with a femoral component that has more valgus in the shaft angle than usual, or in patients with coxa valga, this angular difference could be three to four degrees. In very rare patients who have significant coxa valga or a broad pelvis with a long femoral neck, the angular difference may be seven or eight degrees. In various embodiments, alignment tools can be useful for identifying a range of angles of a cutting guide relative to the anatomic axis, between zero degrees (or less) and eight degrees (or more).

Where the shape and/or size of the anatomical support structure (e.g., bone) significantly varies with resection depth and/or orientation, the proper alignment and positioning of the resection plane will desirably result in a properly sized and/or oriented anatomical support structure to accommodate the implant component. In contrast, an improperly aligned and/or positioned resection plane can result in an improperly sized and/or oriented anatomical support structure, which may not accommodate the implant component and/or may negatively impact the performance and/or durability of the joint replacement procedure.

Various embodiments disclosed herein facilitate the proper positioning and/or orientation of jigs and/or tools that are used in the preparation of anatomical surfaces for accommodating implant components. The various tools and systems described herein desirably utilize a remote or displaced (with respect to the implantation site) location of the patient's anatomy as a reference for alignment of the jigs, tools and/or implant components at a surgical site, thereby significantly increasing the accuracy and repeatability of the surgical implantation procedure.

Various embodiments of tools and methods described herein also enable the adjustment of jigs, tools and/or implant components in a highly accurate manner, allowing a surgeon to “dial in” a desired performance for the implant, such as a desired valgus or varus alignment (or change to an existing alignment) for a knee implant component. In one example, the alignment measurements obtained herein could be utilized to modify the anatomic support surfaces to correct deformities, such as a varus or valgus deformity, in combination with standard implants, which are not typically designed or intended to correct such deficiencies.

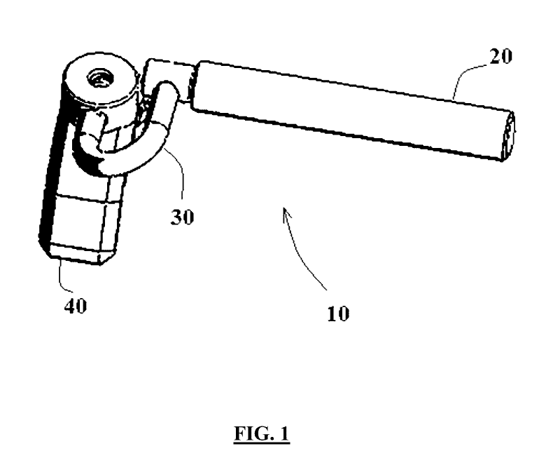

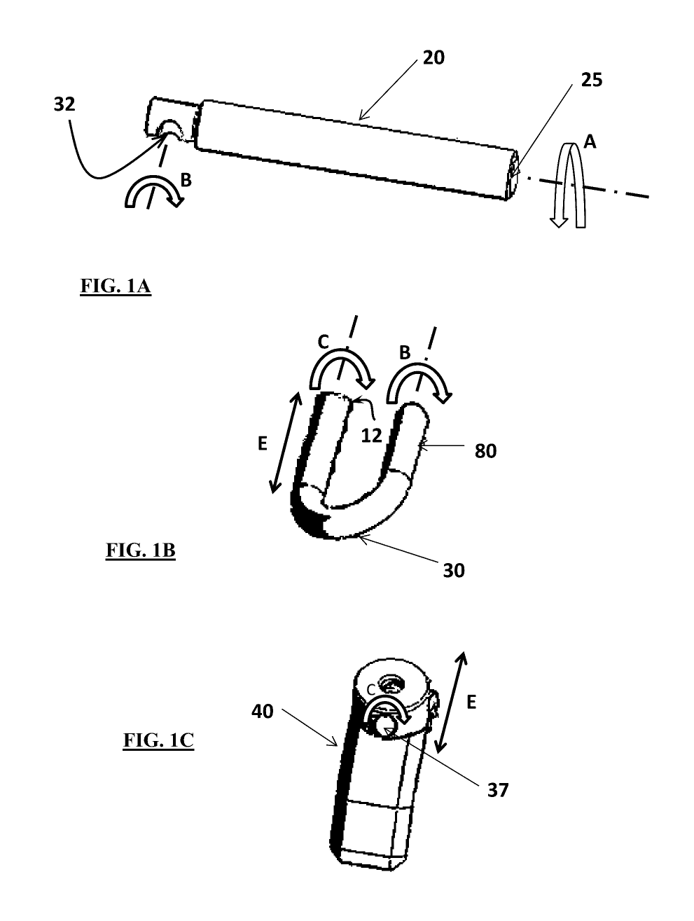

FIG. 1 depicts one embodiment of an alignment tool 10 for use in a knee implantation procedure. The alignment tool may be particularly suited for use with a tibial plateau surgical jig, which can be used to prepare some or all of a patient's tibial plateau for a tibial implant component. In various disclosed embodiments, the surgical jig can be configured to be positioned within a surgical incision inside a surgical field, with the jig positioned directly against a tibial bone of the knee joint. In some embodiments, the alignment tool 10 can be configured to be positioned with at least a portion of the alignment tool 10 outside of the incision, with various components engaging with one or more external skin surfaces (or coverings adjacent thereto) of the patient's extremities.

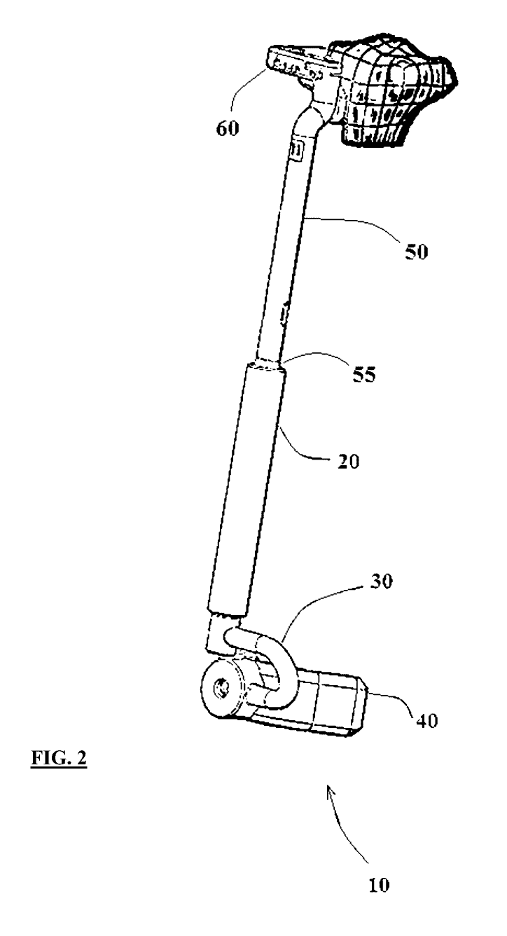

The alignment tool 10 can include a docking sleeve 20, a swivel arm 30 and an ankle clamp 40. The docking sleeve 20 can be sized to slide over and accommodate an alignment rod 50 (or extension alignment rod 55), which can be connected to a surgical jig 60 (see FIG. 2). The alignment rod may be included as part of the surgical jig kit. As shown in FIG. 1A, the docking sleeve 20 can include a generally cylindrical inner bore 25 that is capable of rotating (rotation A) relative to the corresponding alignment rod (see FIG. 2), an arrangement which can facilitate the engagement of these structures. Alternatively, in some embodiments, the docking sleeve 20 can be configured to connect directly to surgical jig 60, or the alignment rod 50 may be configured to connect directly to swivel arm 30.

The swivel arm 30 connects the docking sleeve 20 to the ankle clamp 40. The swivel arm 30 includes a proximal arm portion 80, which can be connected to and capable of rotating (rotation B—see FIGS. 1A and 1B) relative to a circular bore 32 in a distal end of the sleeve 20. The swivel arm further includes a distal arm portion 120, which can be connected to and capable of rotating (rotation C—see FIGS. 1B and 1C) relative to a circular bore 37 formed in a proximal portion of the ankle clamp 40. These arrangements can permit relative movement between the docking sleeve 20 and the ankle clamp 40 in a known, controlled and measureable manner. In some embodiments, the swivel arm 30 will maintain a desired rotational alignment between the sleeve 20 and the clamp 40, yet allow for cephalad/caudad movement of the clamp 40 relative to the sleeve 20 as well as anterior/posterior movement of the clamp 40 relative to the sleeve.

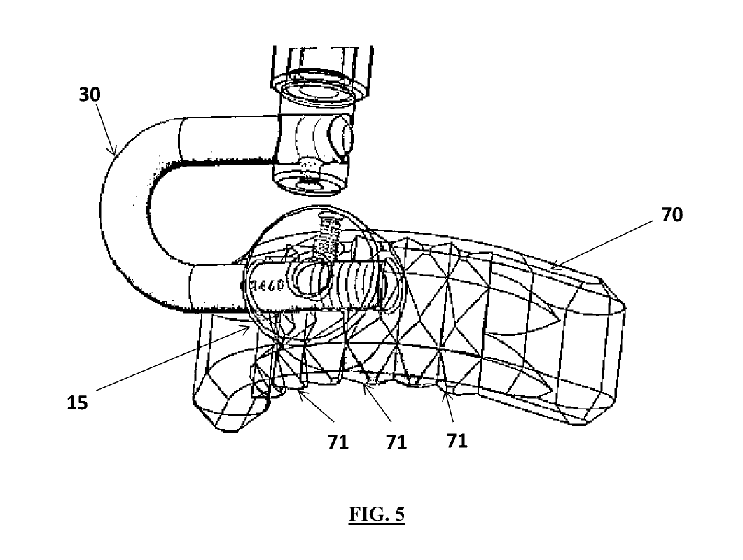

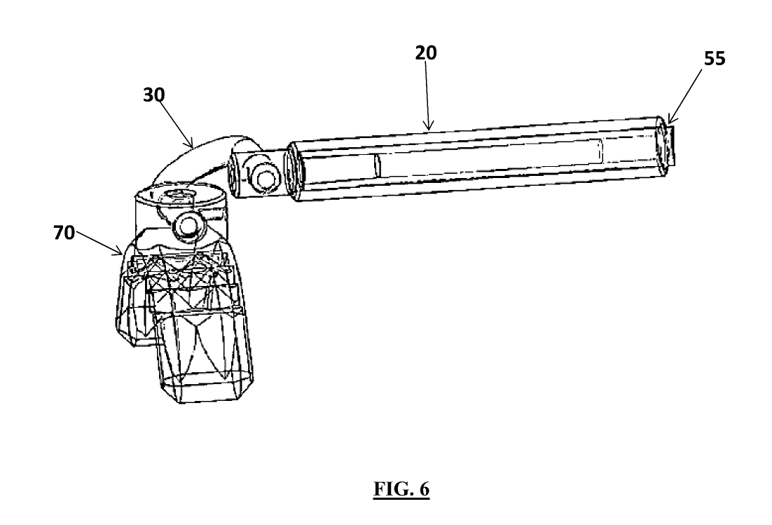

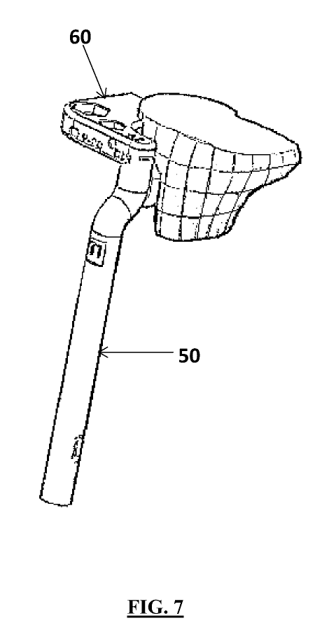

In some embodiments, the ankle clamp 40 can include a textured mating surface 70, which can facilitate placement and securement of the clamp 40 to the targeted patient anatomy, e.g., the patient's ankle. In some embodiments, the mating surface 70 includes a plurality of projections or “teeth” 71 (see FIG. 5) which can engage with a wide variety of surfaces of the targeted anatomy, including skin, surgical skin coverings, bandages and/or surgical drapes.

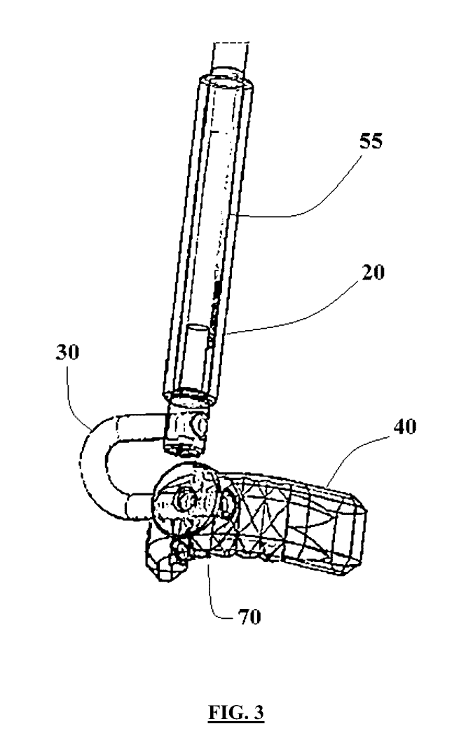

As discussed above, in some embodiments, the docking sleeve 20 is configured to slide over an alignment rod 50, which extends from a surgical jig 60 or other structure. The cylindrical inner bore 25 can be configured to fit over the cylindrical alignment rod 50 with limited clearance, thereby inhibiting relative axial movement (e.g., the relatively tight or close fit desirably results in little or no “toggle” or “slop” between the two engaging pieces) between the sleeve 20 and rod 50 (see FIG. 3). In various embodiments, this sliding or telescoping configuration of the rod 50 and the sleeve 20 can permit the system to accommodate patient legs of varying lengths (i.e., varying distances between the proximal tibia of the knee joint and the tibial crest of the patient's ankle), with additional extension alignment rods 55 being used in some embodiments to accommodate patients with extremely long lower legs. As previously noted, rotation of the sleeve 20 relative to the rod 50 can accommodate various orientations of the rod, which may be due to variations in the patient's anatomy as well as the design and positioning of the various surgical jigs and/or tools used during the surgical procedure (e.g., to accommodate varying sagittal or other slopes that the tibial jig may assume for a variety of reasons).

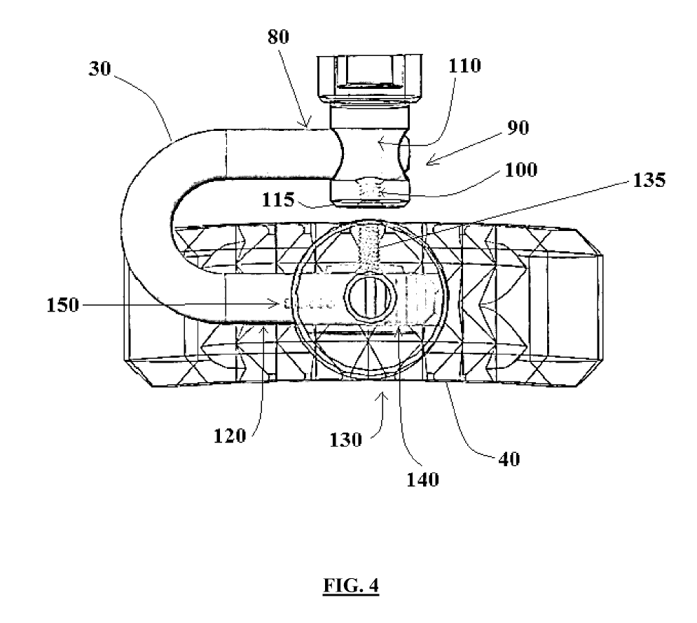

FIG. 4 depicts the swivel arm 30 connecting the docking sleeve 20 to the ankle clamp 40. The proximal end 80 of the swivel arm 30 (adjacent the docking sleeve 20) includes a swivel connection feature 90, which allows the proximal end 80 of the swivel arm 30 to rotate relative to the docking sleeve 20. In this embodiment, a screw 100 interacts with a groove 110 in the swivel arm 30 to secure the proximal end 80 within the bore 32 formed in a fitting 115 (the fitting 115 can be connected to, or a portion of, the distal end of docking sleeve 20), yet allow the swivel arm 30 to rotate relative to the fitting 115 (rotation B) in a controlled and predictable fashion.

A distal end 120 of the swivel arm 30 can be connected to a bore 37 formed in the ankle clamp 40 by a detent fitting 130. The detent fitting 130 can allow rotation of the ankle clamp 40 relative to the swivel arm 30 (rotation C), and further allow controlled translation (i.e., longitudinal displacement) of the swivel arm into and out of the detent fitting 130 of the ankle clamp 40 (translation E of FIGS. 1B and 1C). In this embodiment, the detent fitting includes a hollow detent screw 135 having a detent ball and spring arrangement therein (not shown). The detent screw 135 is positioned adjacent to a series of grooves 140 formed in the distal end 120, with the detent ball interacting with the grooves 140 in a known manner. The longitudinal movement of the swivel arm relative to the ankle clamp can be accompanied by a physical “clicking” sensation and/or audible “click” sound, although a wide variety of connection mechanisms, including locking mechanisms or other connection systems, can be used.

As previously noted, the swivel arm's ability to rotate relative to both the docking sleeve 20 and the ankle clamp 40 in a relatively constrained fashion permits the ankle clamp 40 to be adjusted to accommodate a wide variety of patient sizes and shapes while presenting the mating surface 70 of the ankle clamp 40 towards the patient. In this manner, the tool can provide an accurate measurement of relative alignment along one or more desired planes and/or axes for a wide variety of patient shapes, sizes and/or anatomies.

A series of markings 150 (e.g., numbers, letters, colors, textures and/or other indicators) can be included on the distal end 120 of the swivel arm 30, with one or more of the markings 150 visible on a portion of the swivel arm 30 which extends outside of the detent fitting 130 (see FIGS. 4 and 5). In various embodiments, a similar series of markings can be included on the reverse side of the swivel arm (not shown).

In some embodiments, the docking sleeve 20 of the alignment tool 10 can be slid over an alignment rod 50 connected to a surgical jig 60 (or tool/implant, if desired) (see FIG. 2). The ankle clamp 40 can be positioned against the patient's ankle (or against a drape or other covering of the ankle used during surgery) adjacent to the tibial crest of the ankle (not shown), with the mating surface 70 securing the clamp 40 relative to the surface. As previously noted, the mating surface 70 can comprise an irregular surface of teeth 71 that can mechanically and/or frictionally engage with a surgical drape or other feature on the ankle surface. If desired, the ankle clamp 40 can be adjusted side-to-side (e.g., medially/laterally, anteriorly/posteriorly) by sliding the clamp 40 relative to the swivel arm 30 along the detent mechanism.

Once the alignment tool 10 is properly positioned, the actual alignment of the surgical jig, tool and/or implant relative to the patient's tibial crest/ankle can be determined from the markings 150 on the swivel arm 30. In the embodiment shown in FIG. 4, the marking “0” is visible adjacent the edge of the detent fitting 130, which indicates that the jig (not shown) is aligned parallel to the mechanical axis of the tibia. Other readings in this figure, if visible, could indicate other alignments, such as offsets of 2 mm, 4 mm, 6 mm or 8 mm (or −2 mm, −4 mm or similar measurements) between the jig, tool and/or implant and a line drawn along the mechanical axis of the bone. If desired, the markings could alternatively indicate relative offset angles, such as 0°, 2°, 4°, 6° or 8°, between the jig, tool and/or implant and the mechanical axis of the bone. In the various embodiments described herein, the markings could include measurements of −12 to +12 (e.g., mm or degrees) in single digit or even/odd number increments, or variations thereof.

The alignment readings can be used to accomplish various objectives. For example, the readings may be used to adjust the orientation and/or positioning of the jig to obtain a desired alignment, such as alignment of the jig so that the alignment rod is positioned parallel to the mechanical axis of the tibia in the sagittal plane. Once a desired alignment is achieved, the jig may be secured to the tibia with one or more pins or other attachment devices in a known manner, and the alignment tool removed (if desired).

In some embodiments, the readings may be used to align the jig to an alternative orientation, such as to alter the orientation of a pre-existing standard implant design, to obtain altered performance from the standard implant.

Additionally or alternatively, the readings may be used to select a specified implant and/or spacer device (e.g., a polyethylene tibial tray insert or combinations of inserts) for use in accommodating the measured alignment in a final implant. If desired, the tools and techniques described herein could be used to align other jigs, tools and/or implant components in a similar manner.

In various embodiments, the readings could be used to determine if a given anatomy and/or surgical resection is within a desired margin of error, or if a surgical preparation step (e.g., cutting or drilling action performed by the surgeon) has or will create an incorrect and/or undesirable feature during the surgical procedure.

In various embodiments for aligning a tibial resection plane, the alignment of the tibial cut in the coronal plane can be important for optimal function of the resulting joint replacement implant. Various of the disclosed embodiments can provide for the highly accurate determination of such alignment, while accommodating for significant variations in the anatomical features of the patient, which could include a wide variety of heights and/or weights of the patient. In some embodiments, the alignment of the jig could be parallel to the mechanical axis in the sagittal plane of the bone to be engaged by the jig, although other alignments, such as alignment with one or more anatomical and/or mechanical axes of the extremity, the joint and/or the various bone structures could be used.

In some embodiments, the markings on the reverse side of the swivel arm (not shown) can be utilized to determine alignment of the opposing knee, simply by turning the tool over and rotating the ankle clamp 40 by 180°. The tool can then be utilized as previously described, without requiring disassembly or modification of the tool.

In one or more alternative embodiments, the markings could include both positive and negative indicators, which would indicate potential variation on either side of the reference plane or axis. In another alternative embodiment, the opposing sides of the swivel arm could carry different indicators, with one side having positive indicators and the opposing side having negative indicators. One example of such a system for use with a knee joint could be indicators on one side of the tool to indicate varus variation, with the opposing side indicating valgus variation.

The alignment tool may be disposable and/or sterilizable, depending upon component materials. In various embodiments, the tool could include medical grade nylon or other plastic material, or could include stainless steel or other materials known in the art.

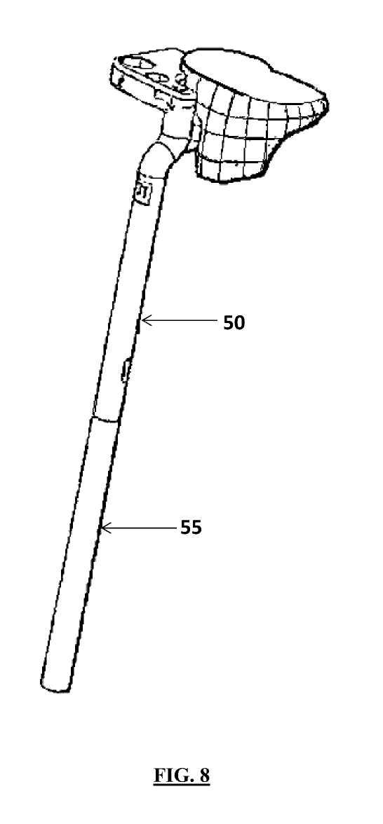

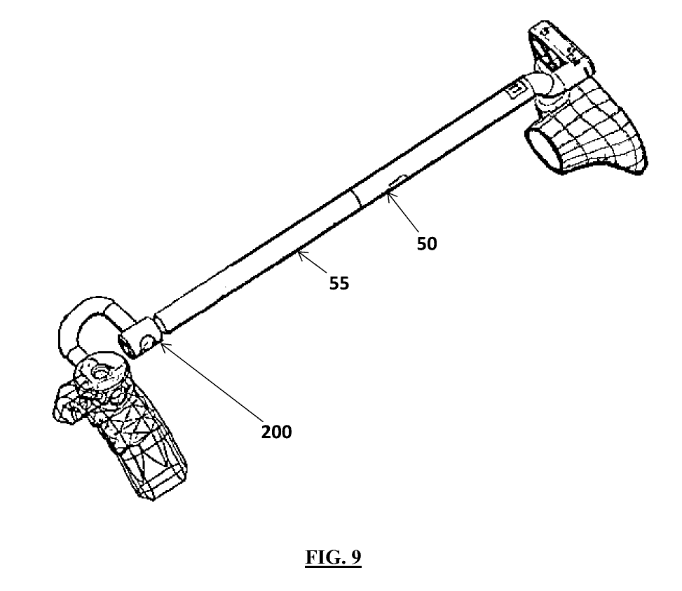

In some embodiments, as shown, for example, in FIGS. 8 and 9, the docking sleeve 20 could include an alternative mechanism for engaging with the alignment rod 50 or alignment rod extension 55. In this embodiment, the sleeve 20 can include a threaded connection fitting 200 or other arrangement (e.g., a tapered fitting or bore) which can connect to a corresponding threaded bore (or tapered bore/protrusion) formed into the end of an extension alignment rod 55 (or alignment rod 50).

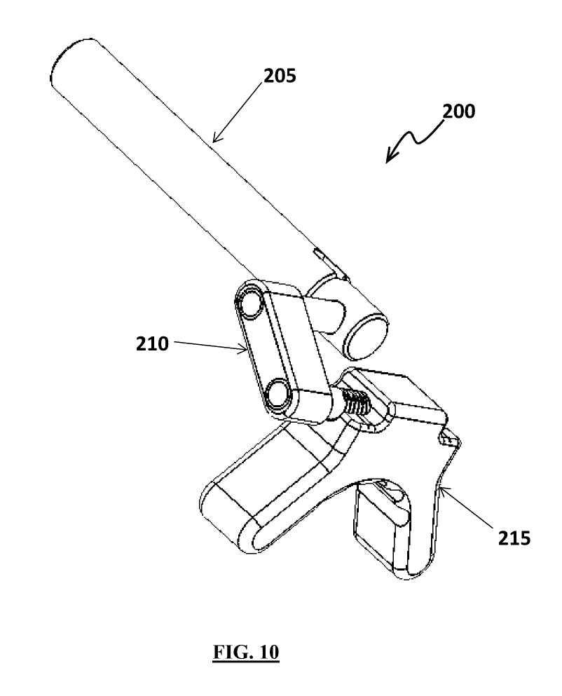

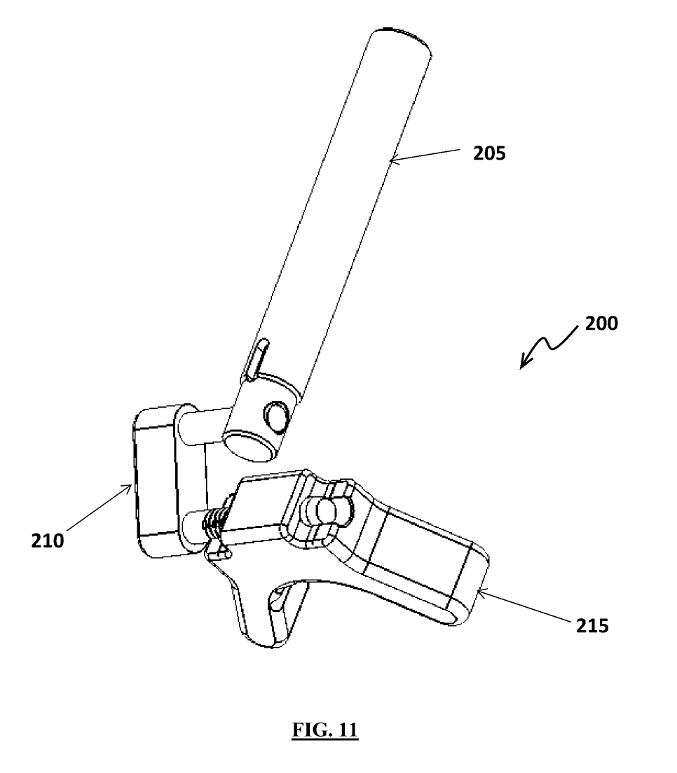

FIGS. 10 through 12 depict various perspective views of another alternative embodiment of an alignment tool 200. The alignment tool 200 can include a proximal section or docking sleeve 205, a swivel arm 210 and a distal section or mating arm 215. Various features of alignment tool 200 may enhance the functioning and/or manufacturability of the alignment tool 200.

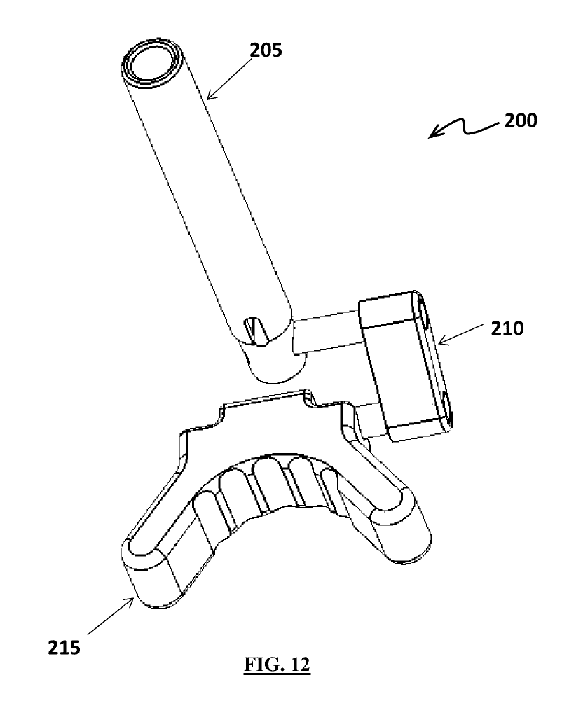

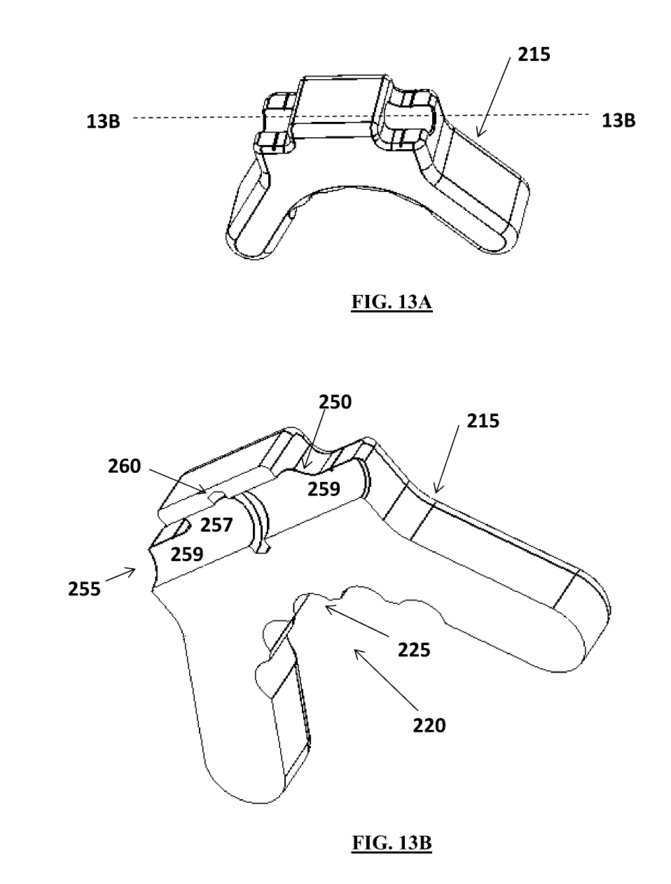

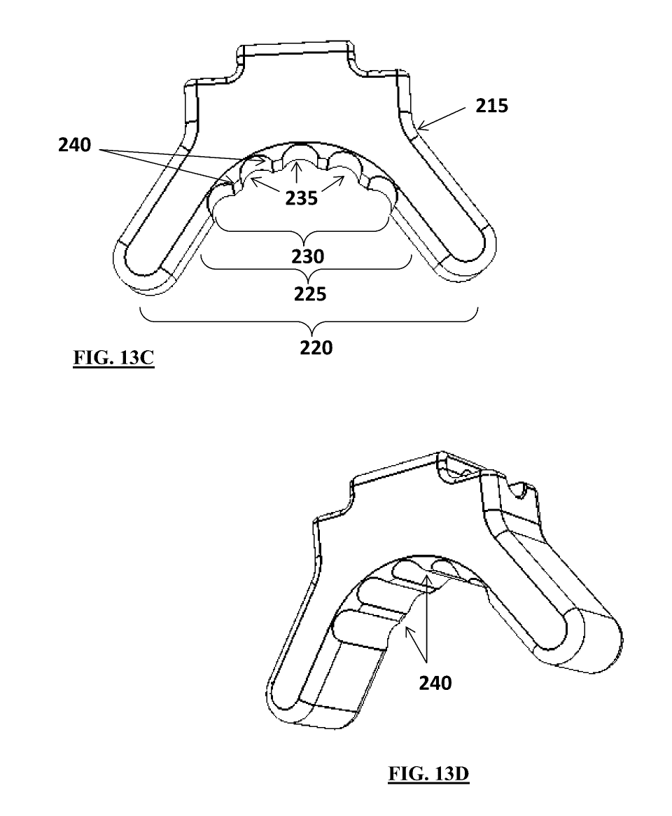

FIGS. 13A through 13D depict various views of the mating arm 215. The mating arm 215 includes an engaging or mating surface 220, which optionally includes a concave section 225 having a textured inner surface 230 configured to engage and/or secure against a patient's anatomical structure of interest and/or covering thereof (e.g., surgical drapes). In some embodiments, the concave section can optionally include a series of scalloped regions 235 that separate a plurality of blunted teeth 240. This arrangement can be configured to provide sufficient frictional or other engagement with the patient's anatomy and/or coverings thereof to secure the mating arm 215 in a desired location.

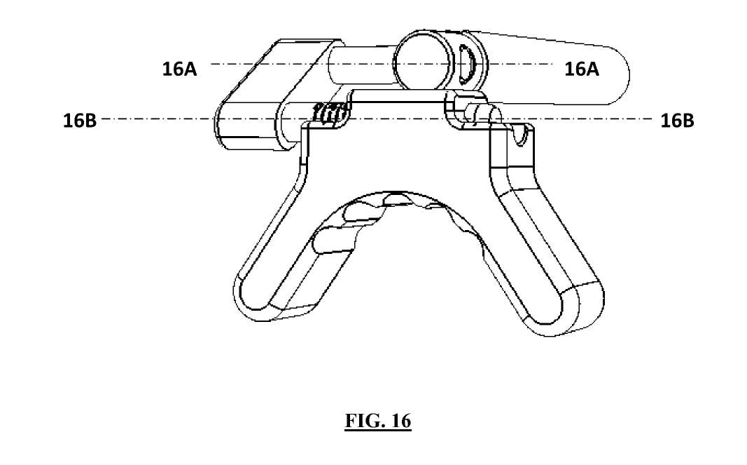

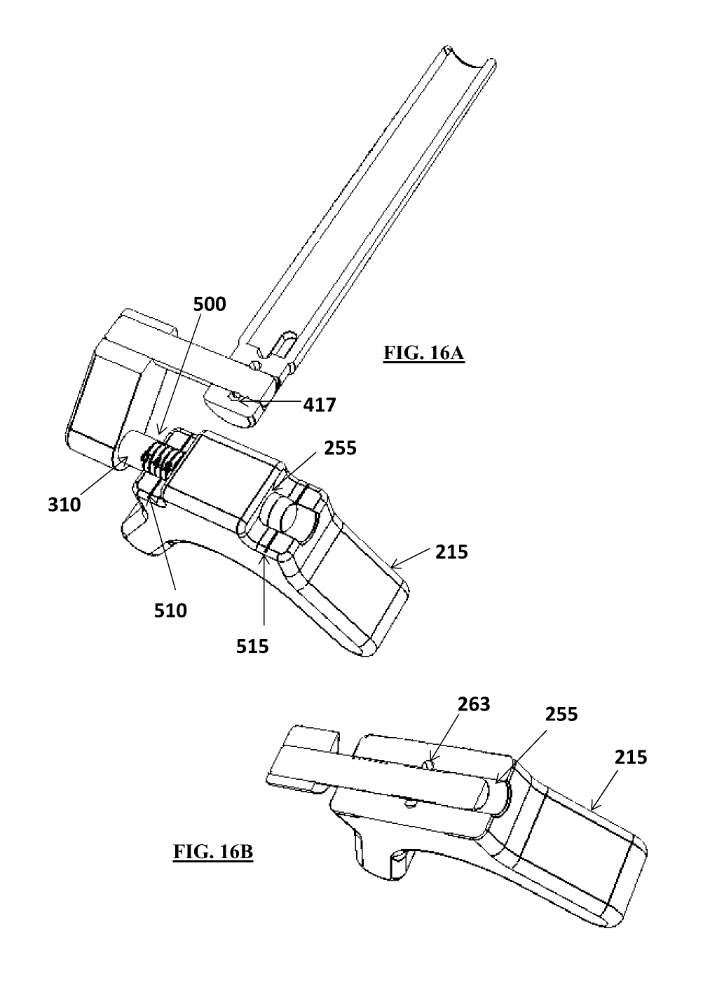

The mating arm 215 further can include a connection feature 250 that facilitates the rotation of the arm 215 relative to the swivel arm 210, and further facilitates longitudinal translation and/or assembly/disassembly of the mating arm 215 relative to the swivel arm 210. As best shown in FIGS. 13A and 13B, the connection feature includes an elongated bore 255, which extends through a portion of the mating arm 215. The bore 255 may include a fully sleeved or “closed” central portion 257 and partially sleeved or “open” sections 259 on each side of the central portion 257. A bore groove or channel 260 can be formed within the wall of the central portion 257, with a canted coil spring 263 (see FIG. 16B) or other engagement feature contained therein. The canted coil 263 spring may be selected with an outer diameter approximate to or slightly larger than the depth of the channel 260, such that some portion of the spring extends outward from the channel 260 into the bore.

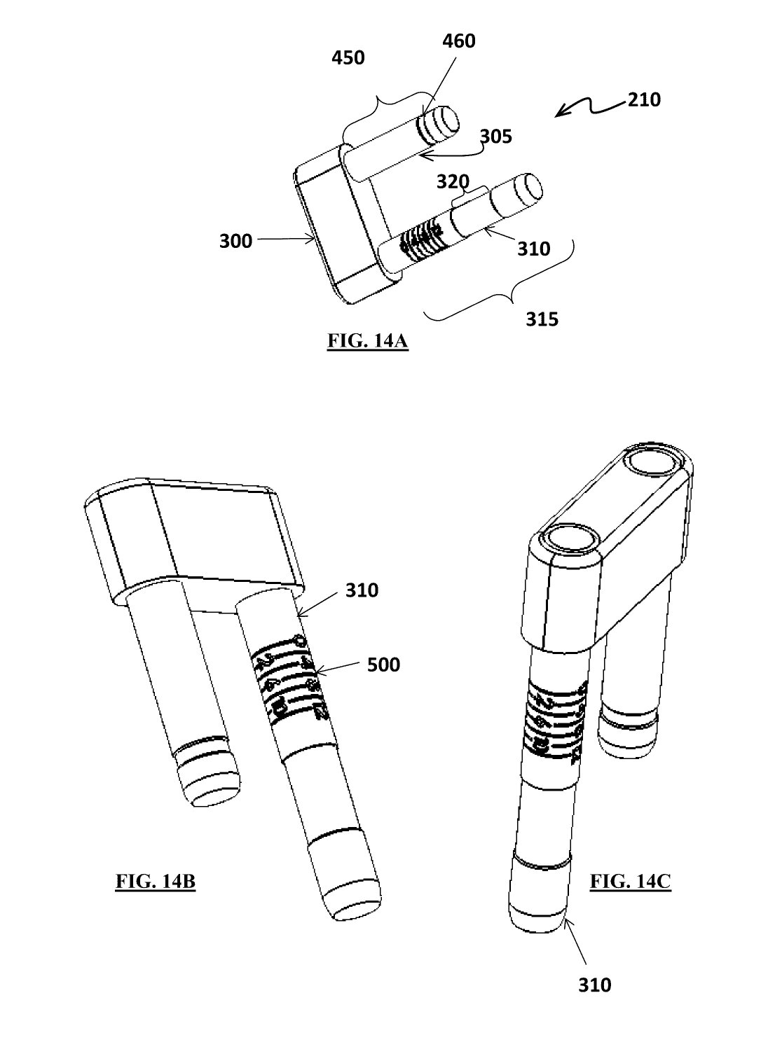

FIGS. 14A through 14C depict various perspective views of a swivel arm 210. The swivel arm 210 includes a central body 300, a proximal swivel arm 305 and a distal swivel arm 310. The distal swivel arm 310 can include a generally cylindrical elongated body 315 having a reduced diameter section 320 positioned along its length. The outer diameter of the arm 310 can be sized and configured to fit within the elongated bore 255 of the mating arm 215 with a relatively tight tolerance, such that arm 310 can rotate and move longitudinally within the bore 255 with little “slop” or “toggle.”

In use, the distal swivel arm 310 can be inserted into the elongated bore 255 of the mating arm 215 in a manner similar to that of the previously described embodiments. The canted coil spring 263 within the bore 255 can be configured to resist insertion of the arm 310 to a limited degree, and secure or “lock” the arm 310 within the bore 255 when it encounters the reduced diameter section 320. The reduced diameter section 320 can be of sufficient width to allow the arm 310 to be displaced longitudinally to a desired degree while the canted coil spring maintains the arm 310 within the bore 255. To remove the arm 310 from the bore 255 (such as when disassembly of the tool is desired), application of an additional, but not excessive, force to withdraw the arm 310 can deform the canted coil spring 263 to a degree sufficient to allow removal of the arm 310 from the bore 255 in a reverse manner to that previously described above for insertion.

In some embodiments, the configuration of the canted coil spring 263 with the reduced diameter section 320 can facilitate the system to allow “infinity longitudinal adjustability” of the arm 310 relative to the bore 255. That is, this configuration can allow the system to be secured in almost any longitudinal position (as opposed to a limited number of set locations), which can greatly increase measuring accuracy and reduce the opportunity for surgical errors.

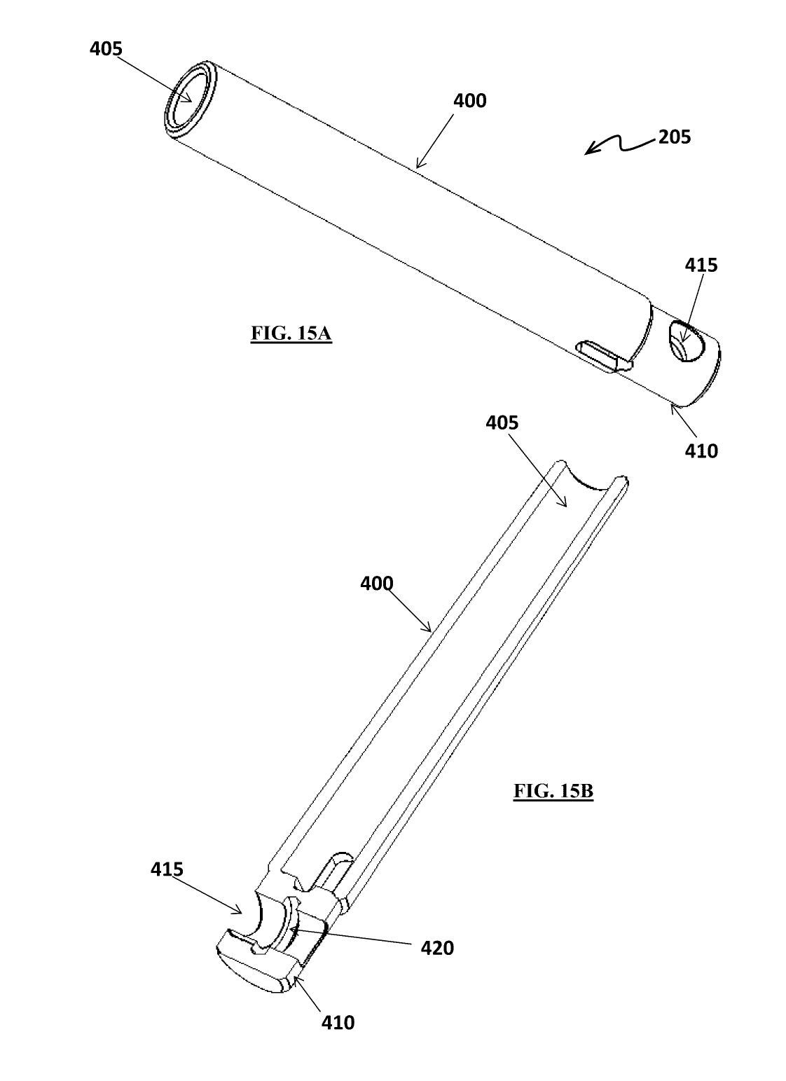

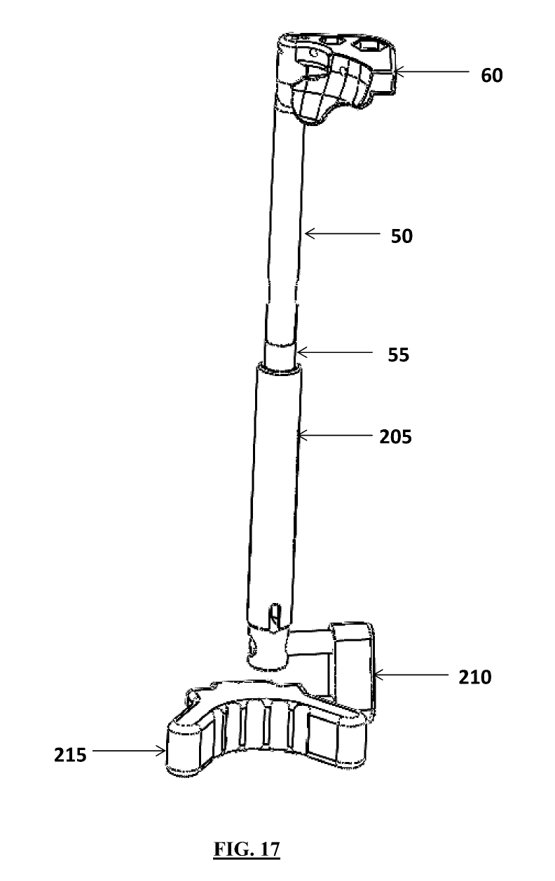

FIGS. 15A and 15B depict views of the docking sleeve 205. The docking sleeve 205 can include a substantially elongated cylindrical body 400 having a longitudinally extending central bore 405 and a distal connecting section 410. The central bore 405 can be sized and configured to receive the cylindrical shaft of a corresponding alignment rod 50 or alignment rod extension 55 (see FIG. 17) that is attached to a surgical jig, tool or implant component. In various embodiments, the bore 405 may allow longitudinal movement and/or rotation of the alignment rod. In other embodiments, the docking sleeve 205 may include a locking or detent mechanism (not shown) that releasably secures and/or frictionally engages the alignment rod within the bore 405 in a desired fashion.

The connecting section 410 of the sleeve 205 can include a generally transverse bore 415 extending through the section 410, with a groove or channel 420 formed within the wall of the bore 415. In a manner similar to that previously described, a canted coil spring 417 (see FIG. 16A) or other engagement feature can be contained within the channel 420, with the canted coil spring 417 having an outer diameter approximate to or slightly larger than the depth of the channel 420, such that some portion of the spring extends outward from the channel 420 into the bore 415.

With reference back to FIGS. 14A through 14C, the proximal swivel arm 305 of the central body 300 includes a generally cylindrical elongated body 450 having a reduced diameter section 460 positioned along its length. The outer diameter of the arm 305 can be sized and configured to fit within the transverse bore 415 of the sleeve 205 with a relatively tight tolerance, such that the arm 305 can rotate within the bore 415 with little “slop” or “toggle.”

In use, the proximal swivel arm 305 can be inserted into the transverse bore 415 of the sleeve 205 in a manner similar to those of the previously described embodiments. The canted coil spring 417 within the bore 415 can be configured to resist insertion of the arm 305 to a limited degree, and secure or “lock” the arm 305 within the bore 415 when it encounters the reduce diameter section 460. The reduced diameter section 460 can be of sufficient width to secure the arm 305 against further longitudinal displacement while the canted coil spring 417 maintains the arm 305 within the bore 415. To remove the arm 305 from the bore 415 (such as when disassembly of the tool is desired), application of an additional, but not excessive, force to withdraw the arm 305 can deform the canted coil spring to a degree sufficient to remove the arm 305 from the bore 415 in a reverse manner to that previously described for insertion.

As best seen in FIG. 16A, the distal arm 310 of the swivel arm can further include various indicia or markings 500. The indicia 500 desirably reflect the amount of longitudinal advancement of the distal arm 310 into the bore 255 of the mating arm 215. In particular, in some embodiments, measuring indicia 510 proximate to a lateral wall of the bore 255 (which is exposed by the partially sleeved or “open” sections 259 on one side of the central portion 257) can be read to indicate the relative angle and/or spacing of the mating arm 215 relative to the sleeve 205. As the distal arm is advanced further into and/or withdrawn further out of the bore 255, different markings will be adjacent measuring indicia 510, reflecting a different angle and/or spacing of the mating arm 215 relative to the sleeve 205.

As can best be seen in FIGS. 14B and 14C, markings 500 can be included on opposing sides of the distal arm 310. In use, each of these sets of markings 500 can be used with a corresponding measuring indicia 510 or 515 for use in measuring opposing limb structures and/or other anatomy as desired by the physician. These markings, and the flexibility of the tool, render the tool reversible for various applications as desired. Where desired, the reversible measuring features of the tool can be mirror-images for use in measuring opposing joint structures and/or other features. In various other embodiments, the reversible features may be particularized for an individual patient's anatomy (which may be dissimilar for opposing limbs) and/or for dissimilar surgical procedures.

In some embodiments, the use of canted coil springs for releasable securement can further facilitate the ability to quickly and easily disassemble the entire tool for storage and/or sterilization (e.g., where the tool is formed of Rydel™ or other autoclavable/sterilizable plastics or materials), as well as quickly assemble the tool (and exchange components and/or reverse features of the tool, where desired) without the need for additional instrument (e.g., screwdrivers). Moreover, failure of the disclosed securement mechanism would be unlikely to generate small parts that could fall into an open wound. In addition, the use of canted coil springs can significantly increase the amount of sterilizable area as compared to detents or other securement mechanisms, significantly reducing the sterilization load incurred by the tool.

In various alternative embodiments, the alignment tool could further include a slope indicator (not shown) or other feature to indicate the relative anterior/posterior slope of the sleeve 205 relative to the mating arm 215. For example, the proximal swivel arm 305 could include indicia (not shown) that would indicate a rotational orientation relative to the transverse bore 415 of the sleeve 205 (with corresponding indicia possibly included on distal connecting section 410). Such an arrangement could be particularly useful where the alignment tool is particularized based on anatomical information (including digital image information) of a particular patient.

If desired, the various embodiments of an alignment tool described herein could be generically sized devices. In alternative embodiments, such alignment tools could be designed to accommodate patient and/or population-specific anatomy. If desired, the markings for such tools could reference a desired alignment for the jig, tool and/or implant, as opposed to variation from a generic or given reference plane or axis.

In various embodiments, the alignment tool can be configured for attachment to the tibial jig and extending between the center of the patient's ankle and proximal to the tibial tubercle. The tool can set the tibial jig to be parallel to the tibia mechanical axis, if desired, and can be constructed to be telescoped between these two reference points. The tibial jig can include a cutting platform or other feature that includes a cutting guide slot formed across the jig to receive, for example, a reciprocating saw blade fitted there through (to cut across the tibia). In one embodiment, the tibial cut plane may be cut at a ten degree (10 DEG) angle below a perpendicular plane to that patient's mechanical axis. In various embodiments, the tibial jig could further include a tibial depth resection guide that is positionable across the proximal tibia end to set a desired depth of tibial resection.

In some embodiments, the alignment tool (or portions thereof) could be designed as patient or population-specific using images and/or other information about the specific patient's and/or population group's anatomy. For example, the ankle clamp could be designed to accommodate a specific patient's anatomy at the ankle, with the image data utilized to estimate the spacing and positioning of the tibial crest relative to the overlying hard and soft tissues (including the overlying skin surface). If desired, the ankle clamp and associated measurement markers could be specifically designed and positioned to reflect an actual location of the tibial crest (and/or the actual anatomic center of the ankle joint) relative to the overlying soft and hard tissues. All or some portion of the alignment systems described herein can be designed and/or selected using patient-specific information (e.g., information from pre-operative image data) to identify relevant anatomical features of an individual patient that may be useful for alignment during the patient's individual surgical procedure, or such alignment tools can be designed in a more generic fashion to be useful for a broader patient population and/or population subset (e.g., gender, age, height, size, race). The tools may also be particularized to a specific type or brand of implant, implant component(s), surgical approach/exposure and/or surgical jig/tool system.

In various embodiments, an alignment tool as described herein can include an alignment head or other feature that is positioned in a desired location and/or orientation relative to one or more of the patient's anatomical features. Depending on the tool design and the chosen reference anatomy, the alignment head can reference and/or align relative to anatomical feature(s) in a non-invasive and/or minimally-invasive fashion. In various embodiments, the alignment head and/or other tool portions can include various measuring and/or adjustment features that provide for highly accurate measuring and/or assessment of relevant anatomical features and associated anatomical relationships. In various related embodiments, the system can include close calibration and/or adjustability of the alignment system to facilitate the planning or modification of an intended surgical operation (e.g., altering of a medial/lateral, anterior/posterior, cephalad/coronal and/or other angulation of a tibial cut plane) to achieve a desired surgical outcome.

In various embodiments, the disclosed systems facilitate the accurate positioning, orientation and shaping of proximal tibial and/or distal femoral surfaces (or other relevant anatomical structures and/or surfaces) to receive components of a knee joint prosthesis attached thereto, such that the attached prosthesis will be properly aligned to function as a natural and/or improved knee joint. Various systems include the ability to determine a patient's mechanical axis with reference to their anatomical axis using an alignment guide that can intersect a desired anatomical structure (e.g., a tibial intramedullary canal and/or an ankle joint complex) in a non-invasive manner and which includes alignment and/or guiding features (e.g., cutting guides) that are attached, connected to or otherwise reference the alignment guide such that shaped bone surfaces created using the instrument will shape the tibia in a desired manner to receive one or more the tibial portion(s) of the knee prosthesis.

Various of the embodiments disclosed herein can facilitate the alignment and implantation of articular implants, including those tailored to address the needs of individual, single patients. Various advantages include, for example, less perforation and/or disruption of healthy bone or other tissue structures, more accurate alignment, better fit, more natural and/or desired movement of the joint, simplification of the surgical procedure, reduction in the amount of bone removed during surgery and/or a less invasive procedure.

The various embodiments described herein can be utilized by surgeons with limited experience in partial and total knee reconstruction surgery, and can facilitate the simple and accurate preparation of tibial bone ends (or other joint structures) to receive an intended knee prosthesis. The various systems described herein can also be utilized to measure and/or assess the alignment (e.g., varus or valgus or other alignment) of a pre-operative knee joint, as well as the alignment of one or more prepared bone surfaces and/or implant components of the joint implant replacement. In various situations, the alignment information may be utilized to alter subsequent surgical steps (e.g., alignment information from tibial cuts may alter the intended femoral cuts) and/or may impel a surgeon to modify a chosen implant component and/or insert (e.g., the surgeon may choose a different sized component and/or alter the thickness of a tibial insert to correct an undesirable varus or valgus alignment of the implant components).

Various embodiments described herein disclose a system for determining a patient's mechanical axis with reference to their anatomical axis, using an alignment guide that references anatomical features in a non-invasive fashion. The system includes one or more elongated and/or extensible support structures that reference “remote” or spaced anatomical location(s) (including anatomical features that are adjacent to, spaced apart from and/or otherwise displaced from the surgical site and/or surgically exposed anatomy to varying degrees) as alignment reference points, thereby facilitating the highly accurate alignment of surgical jigs, tools and/or implant components. The system may also include cutting, drilling or shaping guides that are connected to or otherwise reference the alignment guide features that can be employed in shaping and/or forming a bone structure (e.g., a distal femoral head or a proximal tibial head) to receive a corresponding implant component of a prosthetic joint implant. In various embodiments, the alignment guides may include an adjustable linkage and/or a measurement feature (e.g., static and/or changeable) that facilitates the modification of (1) the intended surgical cuts, (2) the intended placement or orientation of implant components and/or (3) alter the selected implant components and/or inserts during the operative procedure.

While it is possible to obtain anatomical information using invasive means (e.g., physical measurement and/or molding of anatomical features during a surgical procedure), it is generally preferred to obtain information about such anatomical structures using non-invasive methods. Patient anatomical image data is most commonly obtained pre-operatively using non-invasive techniques, including magnetic resonance imaging (MRI), plain film X-Ray, X-Ray Computed Tomography (CT), PET and/or SPECT scans, photo acoustic imaging, thermography, fluoroscopy and nuclear medicine, as well as other non-invasive imaging methods known in the art. Once an image data set has been created, the information can be analyzed and interpreted to determine the features of the patient's anatomical structures. In various embodiments, one or more usable electronic models of at least a portion of a patient's joint can be generated from raw image data. Specifically, the patient-specific raw image data and/or measurements can be used to generate a model that includes at least a portion of the patient's joint. In various other embodiments, the data can display information about the outer margins of various softer tissues such as the connective and articular tissues (e.g., tendons, ligaments and articulating cartilage surfaces) of the joint. The image data could also depict various adjacent musculature within the patient's joint, as well as the surface (i.e., skin) boundary, which could be utilized to determine the location of the desired anatomical structure, reference point and/or reference plane from which the alignment can be measured and/or estimated.

In various alternative embodiments, the alignment tools described herein could be utilized to determine other anatomical and biomechanical axes of relevant bones or other structures. If desired, one or more reference points, measurements, structures, surfaces, features and/or combinations thereof could be selected or derived, and alignments and/or variations thereof could be used in positioning jigs, tools and/or implants to address deformities and/or abnormalities. Various alignment devices and arrangements can be utilized for surgical alteration to the joint, including resection cuts, drill holes, removal of osteophytes, and/or building of structural support in the joint deemed necessary or beneficial to a desired final outcome for a patient.

In various embodiments, the reference points, axes and/or measurements can be applied to any joint or joint surface in the body, e.g., a knee, hip, ankle, foot, toe, shoulder, elbow, wrist, hand, and a spine or spinal joints. The articular implant components described herein can similarly include systems appropriate for virtually any joint of the human body, including a knee joint implant component, a hip joint implant component, an elbow component, an ankle joint implant, a shoulder joint implant component, or a spinal implant component. The implants can also be partial joint-replacement implants, such as a femoral condylar or other joint resurfacing implants.

The invention may be embodied in other specific forms without departing from the spirit or essential characteristics thereof. The foregoing embodiments are therefore to be considered in all respects illustrative rather than limiting on the invention described herein. The scope of the invention should include all changes that come within the meaning and range of equivalency of the description, and are intended to be embraced therein.

What is claimed is:

1. An alignment guide for aligning a patient-adapted cutting instrument for knee arthroplasty surgery of a patient, comprising
a docking sleeve for connecting the alignment guide to a portion of the cutting instrument;
a mating arm having an engagement feature for non-invasively engaging an anatomical structure of the patient; and
a connecting mechanism positioned between the mating arm and the docking sleeve; the connecting mechanism adapted and configured to permit controlled relative movement between the docking sleeve and the mating arm.
